(19) United States
(12) Patent Application Publication
(10) Pub. No.: US 2026/0283111 A1
(43) Pub. Date: Sep. 24, 2026

(54) Device for Insect Stimulation Using Electrodes

(71) Applicants: Nanyang Technological University, Singapore (SG); KLASS Engineering & Solutions Pte. Ltd., Singapore (SG)

(72) Inventors: Hirotaka Sato, Singapore (SG); Huu Duoc Nguyen, Singapore (SG); Duc Long Le, Singapore (SG); Phuoc Thanh Tran Ngoc, Singapore (SG); Bing Sheng Chong, Singapore (SG); Kazuki Kai, Singapore (SG); Rui Li, Singpaore (SG); Qifeng Lin, Singapore (SG); Joseph Wei Yang Wan, Singapore (SG); Bing Hui Terence Goh, Singapore (SG)

(21) Appl. No.: 19/477,068

(22) PCT Filed: Apr. 19, 2024
(86) PCT No.: PCT/SG2024/050255

§ 371 Date: Oct. 20, 2025

(30) Foreign Application Priority Data
Apr. 21, 2023 (SG) 10202301112Q

(51) Int. Cl. A01K 15/02; A01K 67/34

(52) U.S. Cl. CPC A01K 15/02; A01K 67/34

(57) ABSTRACT

A method of controlling a movement of a hybrid robot including a locomotion stimulation device carried by an animal includes monitoring, via a positioning component the locomotion stimulation device, a position and an orientation of the hybrid robot; providing, via a stimulator of the locomotion stimulation device, a stimulus to the animal based on an angular difference between the orientation with respect to a direct path from the position to a predetermined destination to control the movement of the hybrid robot; monitoring, via the positioning component, one or a combination of a displacement, a velocity or an acceleration of the hybrid robot in response to the stimulus; ceasing provision of the stimulus to the animal in response to the one or the combination of the displacement, the velocity or the acceleration being below a corresponding pre-defined minimum so as to allow the animal to roam freely.

### RELATED APPLICATION

This application claims the benefit of priority to the Singapore application no. 10202301112Q filed Apr. 21, 2023, the contents of which are hereby incorporated by reference in their entirety for all purposes.

### TECHNICAL FIELD

The present disclosure relates to neurostimulation apparatus for use with insects and the like, and more particularly to an apparatus with one or more electrodes for delivering neurostimulation.

### BACKGROUND

Insects and the like, if controllable, are potentially useful for accessing small crevices and other difficult to reach places. Neurostimulation or the delivery of electrical signals to certain body parts of an insect can produce a change in the locomotion or behavior of the insect. The conventional method used in the laboratory to attach neurostimulation apparatus to the insect involves surgery, e.g., to implant electrodes into the body of the insect. A less invasive conventional method is to glue the neurostimulation apparatus to a prepared body part. It can be appreciated that surgery and adhesives cause irreversible damage to the insect even if the implanted electrode and/or adhesive is eventually stripped out/off the insect. There is therefore a need for a less damaging alternative.

### SUMMARY

In one aspect, a device includes a base member; and two or more hooks, the two or more hooks being disposed at different sides of the base member, the two or more hooks each being a resiliently flexible hook.

The device may be detachably attachable to the insect by a wholly mechanical coupling of the two or more hooks with the insect.

The two or more hooks may each include: a securing member, the securing member being coupled with the base member; a biased member; and an angled element, the angled element coupling the securing member and the biased member to define a slot between the securing member and the biased member.

In another aspect, a kit for use with one or more insects includes: a plurality of the device, the plurality of the device being of various sizes for use with the one or more insects of various sizes.

In yet another aspect, an insect-computer hybrid system includes: an insect; and one or more devices detachably attached to a respective body part of the insect by a wholly mechanical coupling.

At least one the devices may further include one or more electrodes, each of the one or more electrodes being in operable contact with a respective neuromuscular site of the insect. A selected one of the one or more devices may be coupled to one of the head, the thorax, the abdomen, and the cercus of the insect.

### BRIEF DESCRIPTION OF THE DRAWINGS

Various embodiments of the present disclosure will be described with reference to the following figures:

FIG. 1 is a schematic diagram of a controller system including a backpack for neurostimulation of an insect;

FIG. 2A is a dorsal view of an insect with the backpack secured by a device according one embodiment of the present disclosure;

FIG. 2B is a side view of the device of FIG. 2A;

FIGS. 3A to 3E are schematic drawings of the device according to one embodiment of the present disclosure;

FIGS. 4A and 4B are schematic drawings of a mounting platform;

FIGS. 5A to 5C are images showing a method of attaching the device and mounting platform of FIGS. 3A to 4B to a cockroach;

FIG. 6 is a schematic flow diagram illustrating the method of FIGS. 5A to 5C;

FIGS. 7A to 7D are schematic drawings of the device according to another embodiment of the present disclosure;

FIG. 8 is a schematic drawing showing the device according to yet another embodiment of the present disclosure, as worn by an insect;

FIG. 9A is a schematic drawing of the device of FIG. 8;

FIG. 9B is another view of the device of FIG. 9A;

FIG. 10A is a schematic drawing of the connector of the device of FIG. 9A;

FIG. 10B is a schematic drawing of the connector of FIG. 10A showing an electrically conductive element disposed therein;

FIGS. 11A to 11C are images showing a method of attaching the device of FIG. 9A to the head of a cockroach;

FIG. 12 is a flow diagram illustrating the method of FIGS. 11A to 11C;

FIGS. 13A to 13C are images showing a method of attaching the connector of FIG. 10B to an antenna;

FIG. 14 is a flow diagram illustrating the method of FIGS. 13A to 13C;

FIG. 15 is a schematic diagram of a clip-on connector according to one embodiment of the present disclosure;

FIG. 16 is a schematic diagram of another embodiment of the clip-on connector;

FIG. 17 is a schematic diagram of a spring-loaded connector according to another embodiment of the device;

FIG. 18 is a schematic drawing of another embodiment of the spring-loaded connector; and

FIG. 19 is a schematic diagram illustrating a kit of devices of the present disclosure.

### DETAILED DESCRIPTION

The following detailed description is made with reference to the accompanying drawings, showing details and embodiments of the present disclosure for the purposes of illustration. Features that are described in the context of an embodiment may correspondingly be applicable to the same or similar features in the other embodiments, even if not explicitly described in these other embodiments. Additions and/or combinations and/or alternatives as described for a feature in the context of an embodiment may correspondingly be applicable to the same or similar feature in the other embodiments.

As used herein, the term “and/or” includes any and all combinations of one or more of the associated listed items.

The word “exemplary” is used herein to mean “serving as an example, instance, or illustration.” Any embodiment described herein as “exemplary” is not necessarily to be construed as preferred or advantageous over other embodiments.

As used herein, the singular ‘a’ and ‘an’ may be construed as including the plural “one or more” unless apparent from the context to be otherwise.

The terms “about” and “approximately” as applied to a stated numeric value encompasses the exact value and a reasonable variance as will be understood by one of ordinary skill in the art, and the terms “generally” and “substantially” are to be understood in a comparable manner, unless otherwise specified.

Some processes may be described in terms of steps merely to aid understanding and/or for convenient reference. The delineation between one step and another step may be described as such merely for convenient reference in the present disclosure. It will be understood that in actual implementation there may not be a clear division or transition from one step to another subsequent step. There may be a certain amount of overlap among the steps and/or more than one step may occur or be performed concurrently in time, etc.

As used herein, the term “concurrent”, or “concurrently”, is used loosely to refer to two or more occurrences (or events) that at least partially overlap in time, in which the occurrences may not start at the same time instant and/or end at the same time instant.

Terms such as “first” and “second” are used in the description and claims only for the sake of brevity and clarity, and do not necessarily imply a priority or order, unless required by the context.

For the sake of brevity, the term “insect” is used herein to refer generally to an insect or the like, including but not limited to other arthropods and/or animals having a keratin body part or keratin-like body part. Various embodiments of the device of the present disclosure may be used with other insects, arthropods, and/or animals having a keratin body part or keratin-like body part. The following will illustrate examples of the device with reference to use of the device with an adult cockroach (e.g., Madagascar Hissing Cockroach, etc.) solely to aid understanding and not to be limiting.

The insect 900 may be described in terms of a head 910, a thorax 930, an abdomen 940, and pairs of legs 960. The insect may include one or more pairs of appendages 902, e.g., antennae 920 at the head, cerci 950 near the abdominal segment furthest from the head (rear segments), etc. The insect may have neuromuscular sites in various parts of the body. For example, the insect may have neuromuscular sites under a segmented exoskeleton at the thorax 930 and/or abdomen 940, at the antennae 920, and/or the cerci 950.

### Controller System

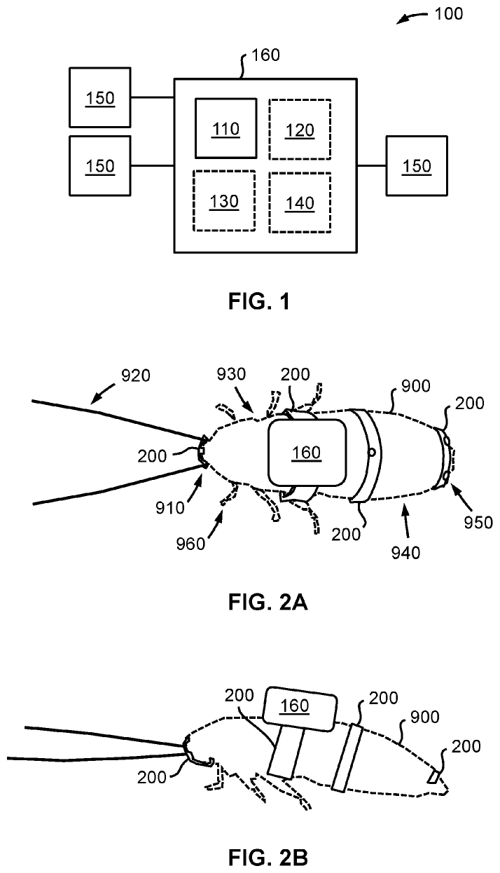

FIG. 1 shows a controller system 100 that can be carried by the insect 900. A part of the controller system 100 may be detachably coupled to the insect in the form of a backpack 160. The controller system 100 in the backpack 160 may include a controller 110 and various other circuit components 140 (including wires 142, conductive tape 144, etc.). The controller system 100 may include one or more electrodes 150 which are attachable to respective body parts of the insect 900. The various components of the controller system 100 are interconnected so that the system 100 may be operable. Each electrode 150 may be provided at a neuromuscular site. The controller system 100 may be operable to deliver neurostimulations to the insect 900 via the one or more electrodes 150. The controller system 100 may be operable for localization of the insect 900, locomotion-related behavior control of the insect, data collection by the insect 900, etc.

The controller 110 may be configured to execute computer-readable instructions stored in a memory. For example, the controller 110 may be pre-programmed. In some embodiments, the controller system 100 further includes a communications module 120. For example, the controller system 100 may be configured for wire communications with external computing devices, base stations, etc., e.g., via a wireless communications module 120.

In some embodiments, the controller system 100 may be powered by a battery pack 130. The battery pack 130 may be disposed in the backpack 160. In other examples, the battery pack 130 may be supplemented with or replaced by a power source, such as but not limited to a piezoelectric generator, a triboelectric generator, or the like.

In some embodiments, the backpack 160 may include the controller 110 and any one or more of the following: a communications module, a battery pack, and a power source.

The controller system 100 may be configured to selectively stimulate or excite one or more neuromuscular sites of the insect 900 via the one or more electrodes 150. For example, the insect 900 may be excited into travelling faster, reversing, turning right, or turning left, etc., in response to one or more stimulating currents delivered at one or more electrodes 150 at various neuromuscular sites.

FIG. 2A is a dorsal view of a schematic drawing of an insect 900 carrying a backpack 160 that is secured to the insect 900 solely by one or more units of a device 200 according to various embodiments of the present disclosure. FIG. 2B illustrates a side view of various examples of the device 200 wearable by worn the insect at different locations along the length of the insect of FIG. 2A. For example, FIG. 2A and FIG. 2B illustrate various examples of the device 200 of the present disclosure attached to the insect 900 at the head 910, the thorax 930, the abdomen 940, and the cerci 950, respectively.

One or more electrodes 150 may be provided at a neuromuscular site at the abdomen 940 using the device 200 according to another embodiment of the present disclosure. Another electrode 150 may be provided at a cercus of the insect 900 using the device 200 according to yet another embodiment of the present disclosure. Another electrode 150 may be provided at an antenna 920 of the insect 900 using the device 200 according to still another embodiment of the present disclosure. Advantageously, the device 200 enables the use of surface electrodes, although other types of electrodes are not precluded from use with the device 200. In some examples, a surface electrode is provided at the second segment of an exoskeleton of the insect 900 to serve as a ground electrode. In some examples, another surface electrode is provided at the sixth segment of the exoskeleton such that a potential difference is provided across the ground electrode and the other electrode at the sixth segment. In some examples, another electrode is provided at the base of an antenna, and a potential difference may be provided across the ground electrode and the other electrode at the base of the antenna.

The device 200 is suitable for detachable attachment with various body parts of an insect 900 by a wholly mechanical coupling, as will be evident from the following description of various embodiments of the device 200. The device 200 is configured to enable a purely mechanical and releasable coupling with a body part. The mechanical coupling may be effected by any one of following actions: snap on, clip on, click on, push through, etc.

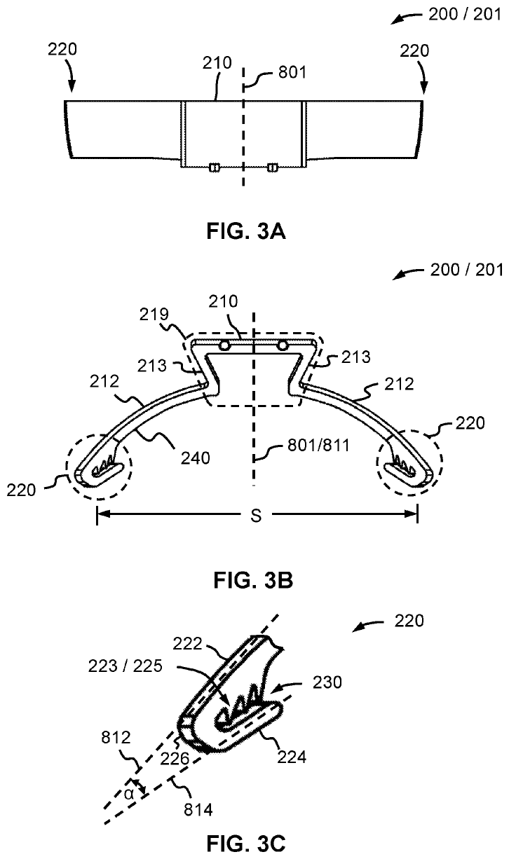

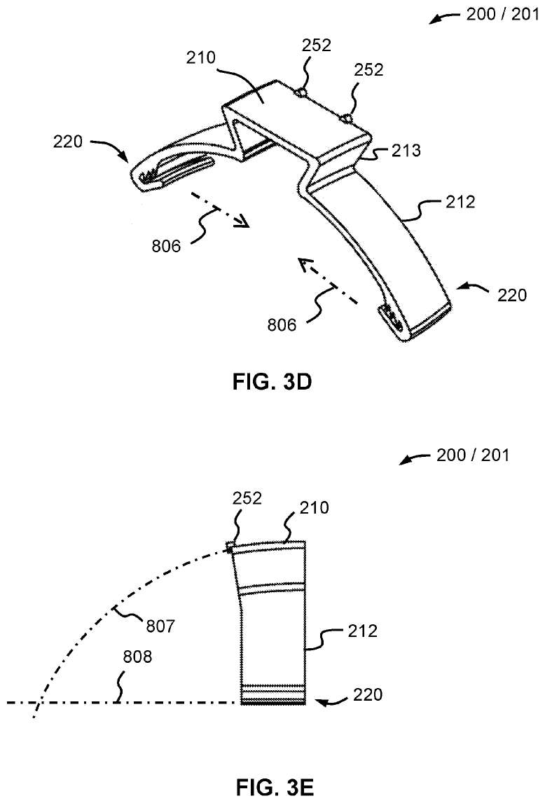

FIGS. 3A to 3E are schematic drawings of a first embodiment 201 of the device 200 suitable for attachment to a body part (such as but not limited to the thoracic region or the thorax 930) of the insect 900.

FIG. 3A shows a top view of the device 200. The device 200 may be configured with mirror symmetry about a plane of symmetry 811 or a line of symmetry 801. The device 200 may be described as having a base member 210 with two or more hooks 220 spaced apart from one another, e.g., the two or more hooks 220 may be disposed at different sides of the base member 210.

FIG. 3B shows another view of the device 200 of FIG. 3A. The base member 210 may be coupled to each of the hooks 220 via an elongate member 212. In the example illustrated, the base member 210 and the elongate member 212 are optionally coupled together via a spacing member 213. The spacing member 213 may be hingedly coupled to at least one of the base member 210 and the elongate member 212.

The device 200 may be an integrated or unitary article fabricated in a single piece from a relatively non-rigid material or a relatively flexible material. For example, the base member 210, the spacing members 213, the elongate members 212, and the hooks 220 may be formed as one unitary article. To aid understanding and not to be limiting, the device 200 may be entirely made of one material such as, but not limited to, plastic or sheet metal.

The device 200 as a whole may be described as a resiliently flexible article. For example, the device 200 may be flexed to deform from a default state to a deformed state by a relatively small deforming force (e.g., applied with fingers or with the aid of tweezers). For example, in the absence of the deforming force, the device 200 tends to resiliently or elastically reverts from the deformed state to the default state. The device 200 may be deformed to increase a span(S) between two hooks 220 of the same device 200.

In some embodiments, among the two or more hooks 220 that extend from different sides of the base member 210, the device 200 may optionally include two hooks that extend from opposite sides of the base member 210. In some examples, the two hooks may be oriented to engage the insect from opposite sides of the insect (such hooks also herein referred to as “opposable pair” for the sake of brevity). In some examples, one of the hooks 220 may form an opposable pair with one other of the hooks 220. In some examples, one of the hooks 220 may form an opposable pair with more than one other of the hooks 220. For example, with respect to the same device 200, a first hook may form a first opposable pair with a second hook, and the first hook may simultaneously form a second opposable pair with a third hook. An imaginary line extending through the points of engagement (each point of engagement being a coupling between the insect and each hook 220 of an opposable pair) would intersect the insect 900 such that the device 200 is prevented from rotating relative to the engaged body part of the insect 900.

When the device 200 is mechanically coupled with the insect 900, an inner surface 240 of the device 200 may be in direct and physical contact with the insect 900.

FIG. 3C is a partial and magnified view of one embodiment of one of the hooks 220. The hook 220 may be contiguous with the base member 210 or indirectly coupled with the base member 210. The hook 220 includes a securing member 222 that ends in a bent or angled element 226, oriented to hook around a body part of the insect. The hook 220 may include a biased member 224. The angled element 226 couples the biased member 224 to the securing member 222.

According to various embodiments of the present disclosure, the angled element 226 may be bent or angled to define an angle of any degree. The biased member 224 is coupled to the securing member 222 via the angled element 226 such that the biased member 224 and the securing member 222 do not extend along respective axes 814,812 that are both parallel and coincidental with one another. In some embodiments, the biased member 224 and the securing member 222 of the same hook 220 define respective axes 814,812 that are non-parallel to one another. In some embodiments, the biased member 224 and the securing member 222 of the same hook 220 define respective axes 814,812 that are parallel but non-coincidental with one another.

In some embodiments, the biased member 224 is disposed at an end of the angled element 226 such that the biased member 224 is disposed generally alongside or angled relative to at least a part of the securing member, such that the securing member 222 and the biased member 224 cooperatively define a slot 230 therebetween. The slot 230 extends along and is defined by the biased member 224 and the securing member 222. The angled element 226 couples the securing member 222 and the biased member 224 together in a resiliently flexible coupling. The height of the slot 230 (or the separation between the securing member 222 and the biased member 224 defining the slot) may be configured to have a default slot height that is smaller than the body part of the insect 900 intended to be received into the slot 230. The resiliently flexible nature of the hook 220 enables: (i) the securing member 222 and the biased member 224 to be pushed apart from a default state to allow the body part to be received into the slot 230; and (ii) the securing member 222 and the biased member 224 to resiliently revert to the default state in which the securing member 222 and the biased member 224 press onto the body part from opposing directions, resulting in a secure and wholly mechanical coupling between the hook 220 and the insect 900. That is, the slot height (H) may be described in terms of the distance or the separation between the securing member 222 and the biased member 224. The hook 220 is configured to provide a variable slot height, for example, by the resiliently flexible nature of the hook 220.

At least one of the biased member 224 and the securing member 222 may include a serrated surface 225 defining at least one side of the slot 230. The serrated surface 225 may be configured as a sawtooth configuration. The serrated surface 225 may include a plurality of protrusions or a plurality of teeth 223. In this example, the teeth 223 are provided at the securing member 222.

The slot 230 is sized to receive body part of the insect 900. In this example, the slot 230 is sized to receive a part of the exoskeleton of the insect 900. When the hook 220 is mechanically coupled with the insect 900, the hook 220 is hooked around an edge of the part of the exoskeleton (the hooked edge). When the hook 220 is mechanically coupled with the insect 900, the securing member 222 and the biased member 224 collectively clamp or grip the part of the exoskeleton in between the securing member 222 and the biased member 224.

The teeth 223 may be oriented to dig into the gripped part of the exoskeleton and resist disengagement of the gripped part (of the exoskeleton) from the hook 220. For example, the teeth 223 may be pointed toward the angled element 226 and away from the “mouth” of the slot 230.

FIG. 3D shows the device 200 from a perspective view. The device 200 may be shaped in an overall generally arcuate shape that provides the hooks 220 on different sides of a body part of the insect 900. The hooks 220 may mechanically engage the insect 900 from opposing sides (e.g., right side and left side of the thorax 930). The resiliently biased mechanical coupling provides forces in opposing directions 806 that tends to tighten the mechanical coupling between the device 200 and the thorax of insect 900.

FIG. 3E shows the device 200 from a side view. The base member 210 may define a surface along a curved plane 807. The angled element 226 may define a line of contact with the edge of the exoskeleton, in which the line of contact is disposed on a flat plane 806.

Referring again to FIG. 3B and FIG. 3D, the device 200 optionally includes a base member 210 that is spaced apart from the body of the insect 900 by one or more of the spacing member 213. In the example illustrated, the base member 210 and two spacing members 213 collectively define a slidable element 219 that has a narrower base and a broader top. The base plate 210 or a part of the slidable element 219 may include one or more of a stopper 252. In some examples, the stopper 252 may be a protrusion at an edge of the base plate 210. In some examples, the stopper 252 may be a protrusion at an edge of the spacing member 213.

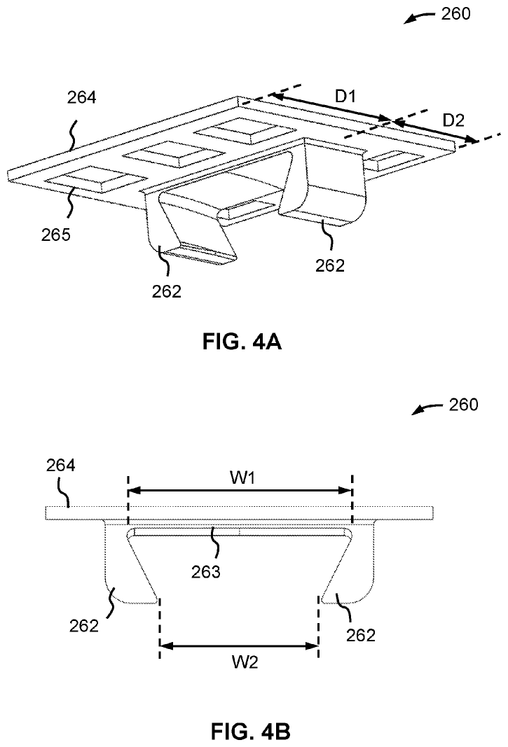

Various accessories may be coupled to the device 200. FIG. 4A is a perspective view of an accessory and FIG. 4B is a front view of one example of an accessory. In the example illustrated, the accessory may include a mounting platform 260 that is detachably coupleable with the base member 210. The mounting platform 260 facilitates the attachment of various items or payloads to the insect 900. In some examples, the mounting platform includes one or more cut-outs 265 in a plate 264. A backpack 160 or other payloads may rest on the plate 264 and tied to the plate at the one or more cut-outs 265.

According to some embodiments, the mounting platform 260 may be slidably coupleable with the base member 210. The mounting platform 260 may include a pair of sliders 262 extending away from a lower face of the plate 265. The pair of sliders 262 may be spaced apart from one another by a first slider width (W1) and a second slider width (W2) corresponding to the broader top and the narrower base of the slidable element 219. The sliders 262 may receive and slidably engage the slidable element 219 until a limiting surface 262 of the mounting platform 262 abuts the one or more stoppers 252 of the device 200. The sliders 262 need not be disposed at a geometric center of the plate 264. For example, the distance D1 and the distance D2 need not be equal to one another.

In some embodiments, besides connecting the mounting platform 260 to the device 200, the sliders 262 prevent deformation or excessive deformation of the base member 210 and the spacing members 213, so that the span(S) between two hooks 220 is constrained. This effectively locks the span(S), making the device 200 attach firmly to the insect 900.

In some embodiments, the backpack 160 may be directly coupled with the base member 210 without the aid of an intervening detachable mounting platform. In some embodiments, the backpack 160 and the base member 210 are detachably coupled. The backpack 160 in such examples may serve to provide a stiffening effect similar to the sliders 262 of the mounting platform 260, to improve firm attachment to the insect 900.

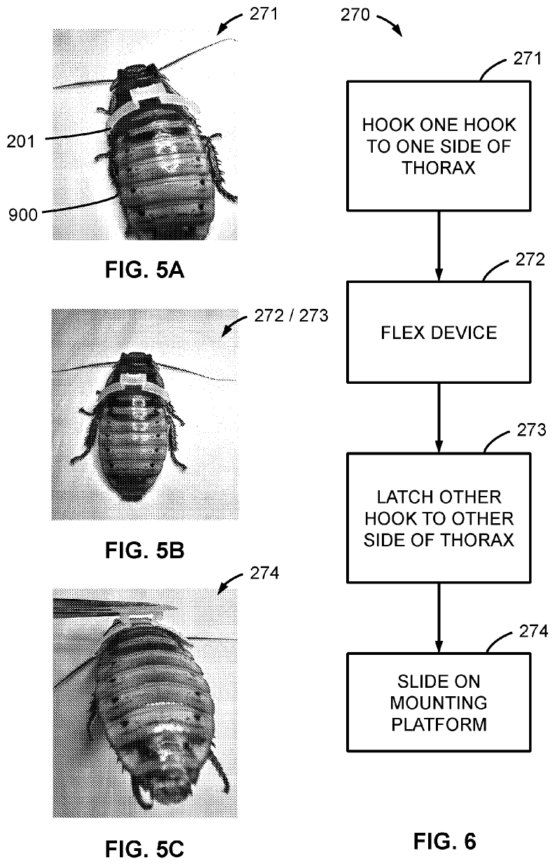

A method 270 of equipping an insect with the device 200 of the first embodiment 201 will be described with reference to FIG. 5A to FIG. 5C, as well as FIG. 6. FIG. 5A to FIG. 5C are images showing various stages of the method 270. FIG. 6 schematically illustrates the method 270 in the form of a flow chart.

In a step 271, a first of the two hooks is slid onto one side of the thorax of the insect 900. For example, this may involve slipping the biased member of one of the hooks under one side of a thoracic segment such that a part of exoskeleton at a thoracic segment is received between the securing member and the biased member.

In step 272, the second of the two hooks is gently extended or flexed to increase the span between the two hooks.

In step 273, the second of the two hooks is made to engage the opposite side of the thoracic segment. For example, with the first of the two hooks engaged at one side of the thoracic segment, the second of the two hooks may be looped to slip the biased member of the second of the two hooks under the other side of the thoracic segment. The two hooks grip opposite sides of the body part of the insect.

In step 274, the sliders of the mounting platform is aligned with the slidable element of the device. The mounting platform may be made to slide onto the base member until the limiting surface abuts the stoppers and is prevented from sliding further.

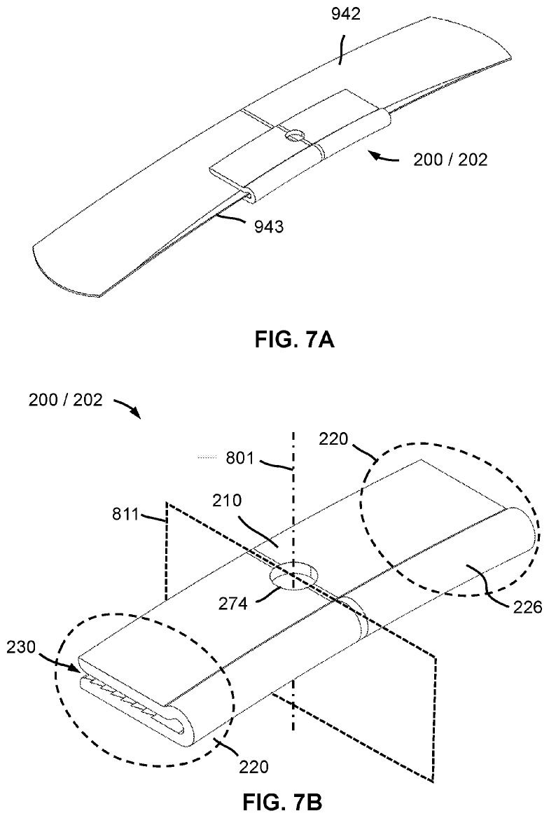

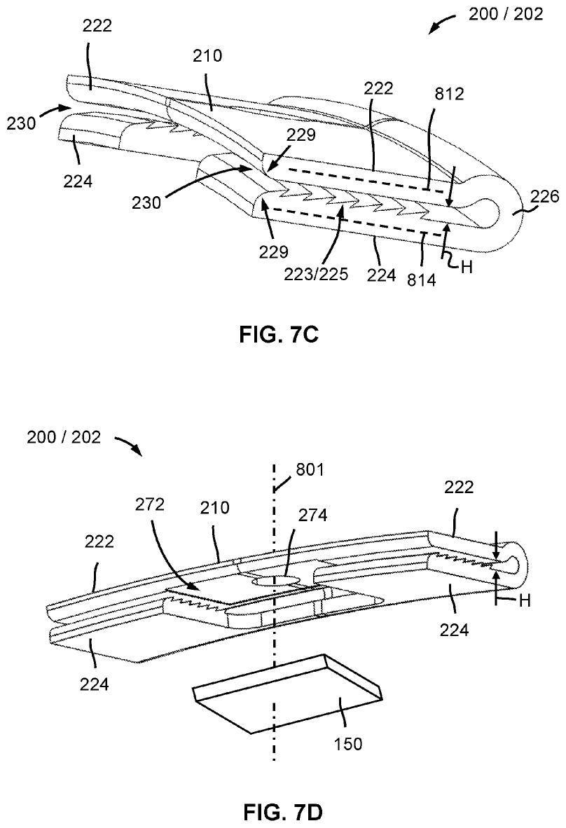

FIG. 7A is a schematic perspective view showing a second embodiment 202 of the device 200 coupled with a segment 942 of the exoskeleton of the insect 900. The segment 942 may be an abdominal segment from the abdominal region of the insect. The second embodiment 202 of the device 200 may be used at other parts of the insect besides the abdomen.

The device 200 may be configured with mirror symmetry about a plane of symmetry 811 or a line of symmetry 801. As shown in FIG. 7B and FIG. 7C, the second embodiment 202 of the device 200 includes two hooks 220. The two hooks 220 are spaced apart from one another (by a span) to mechanically couple the same segment 942 at two different points or locations. In this example, the two hooks 220 are disposed at different sides of a base member 210. The two hooks 220 are similarly oriented relative to the base member 210 to engage the segment from the same edge of the segment. Similar to the first embodiment 201, the second embodiment 202 of the device enables a wholly mechanical coupling with a body part of the insect.

Each of the two hooks 220 includes a securing member 222 and a biased member 224. The securing member 222 and a biased member 224 may extend from different ends of an angled element 226. The securing member 222 and a biased member 224 may extend alongside one another to define a slot 230, in which the slot 230 is configured to receive and resiliently engage a segment 942 of the exoskeleton. When the hook 220 of this embodiment is mechanically coupled with the insect 900, the hook 220 is hooked around an edge of the part of the exoskeleton (e.g., the hooked edge 943 in FIG. 7A). When the hook 220 is mechanically coupled with the insect 900, the securing member 222 and the biased member 224 collectively clamp or grip the part of the exoskeleton in between the securing member 222 and the biased member 224.

Optionally, at least one of the biased member 224 and the securing member 222 may include a serrated surface 225 defining at least one side of the slot 230. The serrated surface 225 may be configured as a sawtooth configuration. The serrated surface 225 may include a plurality of protrusions or a plurality of teeth 223. In this example, the serrated surface 225 is provided at the biased member 224. The teeth 223 may be oriented to dig into the gripped part of the segment 942 and resist disengagement of the gripped part (of the segment 942) from the hook 220. For example, the teeth 223 may be pointed toward the angled element 226 and away from the “mouth” of the slot 230. For example, the hook 220 may be configured with pointed edges that are angled to ensure easy attachment to the edges of an abdominal segment of the insect 900.

As shown in the perspective view of FIG. 7C, the device 200 may be shaped in an overall generally arcuate shape that provides the hooks 220 at different locations along one edge of the segment 942 of the insect 900. The hooks 220 may mechanically engage the insect 900 from two or more spaced apart locations to minimize rotation of the device 200 relative to the segment 942 for a more secure coupling. The resiliently biased mechanical coupling provides forces that tend to tighten the mechanical coupling between the device 200 and the thorax of insect 900. The slot 230 is configured to be narrower than the thickness of segment 942 so that, in assembly, a pressure force securely holds each of the hooks in place. The slot height (H) of the slot 230 (e.g., the separation between the securing member 222 and the biased member 224) may be configured to be smaller than the thickness of the abdominal segment, creating a pressure force that securely holds the hook 220 in place after attachment. Optionally, the securing member 222 and the biased member 224 may each have a respective tapered or chamfered tip 229 at the mouth of the slot 230 to facilitate slotting an exoskeleton segment through the mouth and into the slot 230.

Each of the hook 220 may be shaped with the biased member 224 and the securing member 222 of the same hook 220 defining respective axes 814,812 that are parallel but non-coincidental with one another.

FIG. 7D is a perspective view of the second embodiment 202 of the device 200 exploded along a line of symmetry 801. The device 200 may define a cavity or a recess 272. A surface electrode 150 may be disposed in alignment with the recess 272. A hole 274 may be defined at the recess 272 to accommodate one or more wires and/or conductive tape connecting the surface electrode 150 with the rest of the controller system 100 (e.g., the backpack 160).

The device 200 and the contacted body part of the insect 900 may thus be correspondingly in pressed abutment with one another. As a result, the one or more surface electrodes 150 will correspondingly engage respective neuromuscular sites of the insect 900. For the sake of brevity, as used herein, reference to two items being “in pressed abutment” may be understood to describe the two items being in abutment and simultaneously being pressed or pushed toward one another, forming a direct contact with one another. The resiliently flexible device 200 with two or more hooks 220 enables secure and direct contact via purely mechanical coupling, e.g., without the need for adhesive to be applied between the device 200 and the insect 900. As used herein, “direct contact” can be understood to refer to an absence of an adhesive between the device 200 and the body of the insect 900. While the device 200 does not preclude the application of a dry adhesive (e.g., tape) or a liquid adhesive (e.g., glue) between the contact surface and the insect, the device 200 of the present disclosure renders the application of any adhesive to the insect unnecessary for a proper and/or useful attachment of the device to the insect. The device 200 may also be described as providing a dry mechanical surface attachment with the insect. Advantageously, the device 200 can form a secure attachment to the body part of an insect solely by the releasable mechanical coupling described herein, without the aid of adhesives or other non-mechanical coupling.

### Device With at Least One Connector

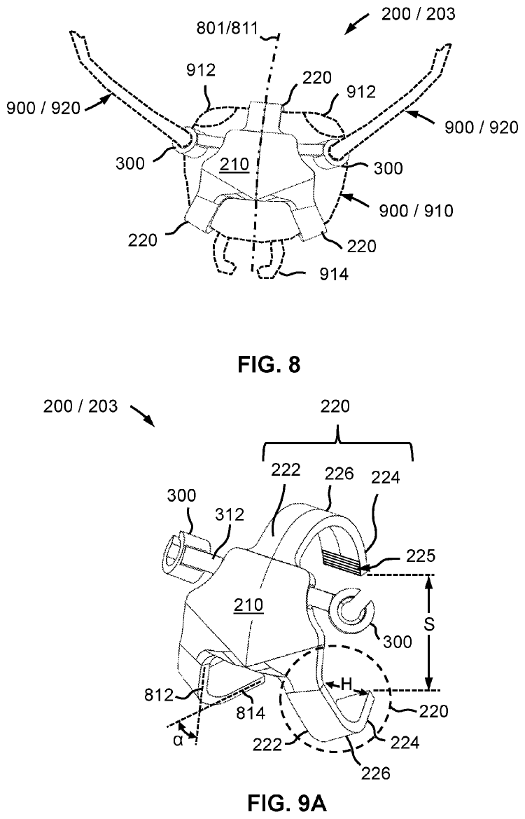

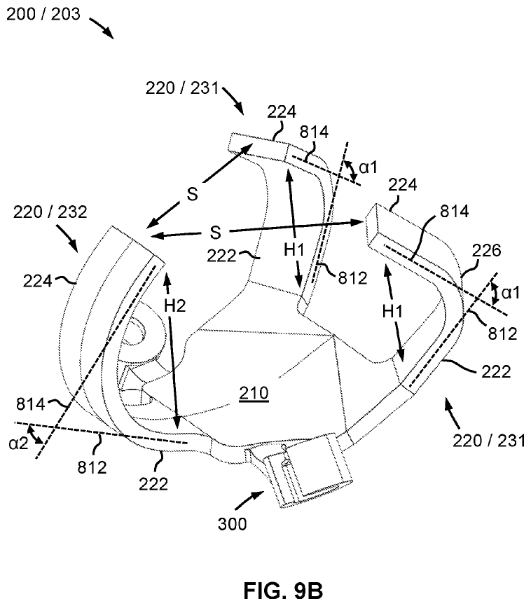

FIG. 8 is an example of a third embodiment of the device 200 showing the device 200 secured to the head 910 of the insect 900. FIG. 9A and FIG. 9B are perspective views of the device of FIG. 8. In these examples, the device 200 includes at least one connector 300, or preferably two connectors 300, that is/are detachably attachable to small or narrow body parts by a wholly mechanical coupling. Examples of the small or narrow body parts may include but are not limited to various appendages 902, such as antennae 920, legs 960, cerci 950, etc.

It can be observed that surface area available at the head 910 of the insect 900 is relatively limited. Advantageously, the device 200 can be securely coupled to the head 910 of the insect 900 despite being limited to a relatively small footprint. Advantageously, the device 200 can be securely coupled to the head 910 without the use of adhesives. In this aspect, the device 200 is suitable for attachment to the small insect head 910 without risk of adhesives getting into the eyes 912 and/or mouthparts 914 of the insect 900 (which may adversely affect the health and performance of the insect 900).

The option to use the present device 200 without involving the use of adhesives on the antennae is a significant benefit to the insect. It can be appreciated that the antennae are fragile but important sensing organs. If adhesives are applied to the antennae to glue on an electrode, great care must be taken to dissolve the glue before removing the electrode lest the antenna is damaged by forcibly stripping the electrode and the adhesive off the antenna.

Preferably, even with the device 200 attached to the insect head 910, the mouthparts 914 of the insect 900 remain unencumbered and free to carry out feeding activities. For example, the hooks 220 may be oriented to contact the surface of the insect head 910 where the hooks 220 would not interfere with the eyes 912 or the mouthparts 914. In some examples, the device 200 may be described as a “helmet” or a “face mask” when the device 200 is “worn” by the insect 900.

The example of the device 200 illustrated in FIG. 9A and FIG. 9B includes a base member 210 and two or more hooks 220 extending from different sides of the base member 210. The device 200 may include two or more hooks 220 disposed at different sides of a base member 210 and extending in different directions. The hooks 220 of the same device 200 may be spaced apart from one another. The hooks 220 may be oriented to confer the device 200 with a mirror symmetry about the plane of symmetry 811 or the line of symmetry 801. Optionally, the device 200 may include three hooks 220.

The device 200 may be formed and shaped so that it exhibits a degree of resiliency or elasticity. For example, the hooks 220 may be resiliently coupled to the base member 210. For example, the hooks 220 may be made of a resiliently flexible material. For example, the base member 210 may be made of a resiliently flexible material. The resilient nature of the hooks 220 may be provided by using a suitable material for the fabricating the device 200. For example, the entire device 200 may be stamped from a piece of stainless steel or aluminum. Alternatively, the device 200 may be 3D printed using a polymeric material. For example, one or more of the hooks 220 and/or base member 210 may be flexed or elastically deformed to enlarge a span(S) between the hooks 220 to receive a part of the body of the insect 900. In the absence of the deforming force(s), the base member 210 and/or hooks 220 spring back or elastically revert to the default state and so reduce the span between the hooks 220. The device 200 is resiliently flexible to provide a variable span between the hooks 220. The hooks 220 cooperate to provide a wholly mechanical coupling to a body part of the insect 900 (e.g., to the head 910 of the insect 900) such that the device 200 is detachably attachable to the insect 900. Optionally, an inner surface of the biased member 224 is configured with a serrated surface 225 to improve a frictional engagement with the contacted part of the insect 900.

A receiving space may also be described in terms of a three-dimensional space that is partially “caged” by the base member 210 and the hooks 220, as shown in FIG. 9B. As illustrated, the device 200 may also be described as a resiliently flexible or elastic article with at least two hooks 220 that are resiliently biased to provide opposing grasping forces. For example, two or the hooks 220 may form two opposable hooks. Two opposable hooks may be oriented in opposite directions to one another.

Each hook 220 of the device 200 may include a securing member 222 coupled to the base member 210 at one end, in which another end of the securing member 222 extends to a biased member 224 via an angled element 226. Optionally and preferably, each one of the hooks 220 may be integrally formed with the base member 210 and made of a material that confers resilient flexibility to the device 200. The biased member 224 and the securing member 222 of the same hook 220 define respective axes 814,812 that are non-parallel to one another. The resiliently flexible nature of the hooks 220 provide a variable slot height between the securing member 222 and the biased member 224.

In some examples, as shown, the device 200 includes two lower hooks 220/231 disposed at a lower part of the base member 210 and an upper hook 220/232 disposed at an upper part of the base member 210. The upper hook 220/232 generally opposes the lower hooks 220/232. Each of the lower hooks 220/231 and the upper hook 220/232 may form a pair of opposable hooks defining a span(S) between the opposable hooks. Each of the hooks 220 may include an angled element 226 extending between a securing member 222 and a biased member 224. The angled element 226 may define a curvature or an angle (α1,α2) of the hook 220. Referring to one hook 220, the securing member 222 may generally define an axis 812 and the biased member 224 may generally define an axis 814, with a curvature or an angle (e.g., α1, α2, etc.) defined by the respective axes. The angled element 226 may dispose the biased member 224 spaced apart from the securing member 222, such that a slot 230 with a slot height (e.g., H1, H2, etc.) defined between the biased member 224 and the securing member 222.

In some examples, the two lower hooks 220/231 may be similarly angled (α1) to define a similar slot height (H1). In some examples, the upper hook 2201/232 may be differently angled (α2) to define a different slot height (H2) from that of the lower hook 220/231.

### Connector

The device 200 in this example includes a pair of connectors 300 extending from the base member 210. Each of the connectors 300 may be coupled to a respective antenna 920 of the insect 900. Each of the connectors 300 provides detachable mechanical clip-on coupling with the respective antenna 920.

The connector 300 may be coupled to the base member 210 via a support element 312. The support element 312 may be configured as an elongated element with one end coupled to the base member 210 and another end coupled to the connector 300. The connectors 300 may exhibit a limited degree of flexibility in their respective positions relative to the base member 210.

The connectors 300, the securing member 222, and the hooks 220 may be formed as a unitary or integral article, so that the relatively tiny connectors 300 will not be loose pieces that are easily lost. Even if a connector 300 inadvertently disengages from an antenna 920, the connector 300 will not drop off the insect completely, but would remain as part of the device 200. The base member 210 and the hooks 220 help keep the connectors 300 spaced apart and positioned near the antenna 920. The base 210 and the support elements 312 may be configured to position each of the connectors 300 near the base of the antennae 910. The relatively small connectors 300 can be kept attached to the head 910 of the insect with the comparatively larger hooking forces provided by the comparatively larger hooks 220.

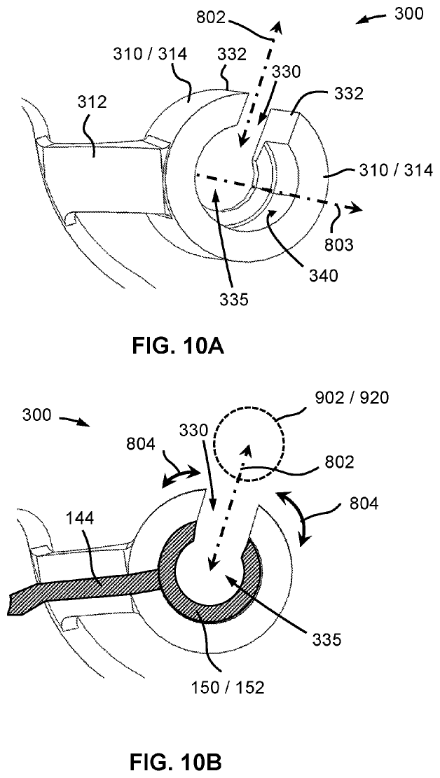

FIG. 10A and FIG. 10B are magnified views of the connector 300 according to some embodiments of the present disclosure. The connector 300 may include connector members 310 that collectively provide a resilient coupling for a releasable engagement with an appendage 902, such as but not limited to an antenna 920.

The connector members 310 of the connector 300 may include a connector securing member 312 and two gripping members 314. The two gripping members 314 extend from the connector securing member 312 in different directions, and the two gripping members 314 are angled (e.g., curved, bent, folded, hinged, etc.) to provide respective connector distal ends 332 that oppose one another. The connector distal ends 332 are spaced apart to form a gap 330 of a variable gap width or gap size. The gap 330 leads to a connector space 335 that may be partially defined by surfaces of the gripping members 314 opposing one another and/or may be partially defined by surfaces of the connector securing member 312 and one gripping member 314 opposing one another.

The connector 300 may be configured such that the displacement bringing the connector distal ends 332 closer to one another is enabled by the resiliency characterizing the connector 300.

### Method

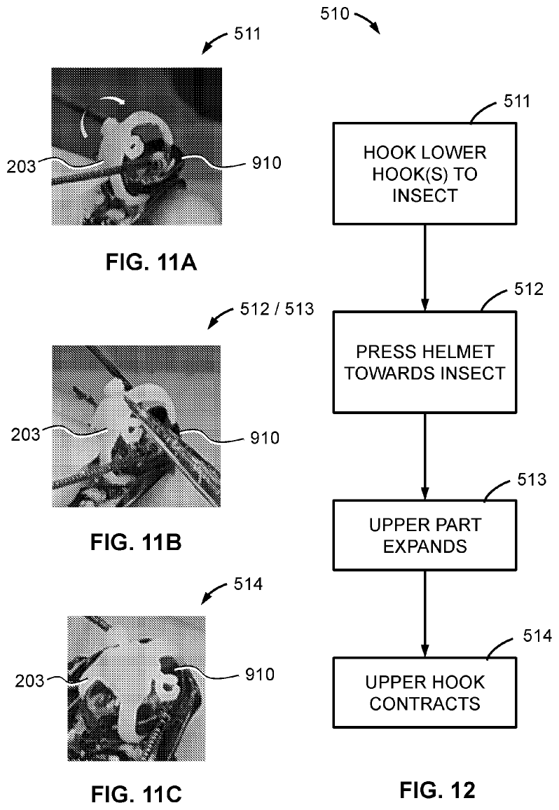

A method 510 of equipping an insect with the device 200 of the third embodiment will be described with reference to the images of FIG. 11A to FIG. 11C and to the schematic diagram of FIG. 12.

For the sake of brevity, the third embodiment of the device 200 may also be referred to as a helmet 203. The helmet 203 can be attached to the head 910 of the insect 900 via wholly mechanical coupling. Advantageously, the helmet 203 can be detached from the head 910 of the insect 900 without a need to dismantle, dissolve, or disintegrate the helmet 203. Beneficially, the helmet 203 can be attached to or detached from the insect 900, all without the need to subject the insect 900 to surgical or invasive procedures.

In step 511 of the method 510, a lower part of the helmet 203 is hooked onto one side of the head 910 of the insect 900. The lower part of the helmet 203 may be one or more hooks 220. As shown in FIG. 11A, two of the hooks 220 are hooked onto a lower facial part of the insect 900.

In steps 512, a force is applied on the base member of the helmet 203 towards the insect's head 910.

In step 513, the base member 210 expands (e.g., increase in the span between the upper hook and the lower hooks) in response to the applied force. This results in an upper part of the helmet 203 expanding and accommodating the shape of the insect's head 910. A slight expansion is found to suffice.

In step 514, in response to the expansion in step 513, the helmet 203 contracts (e.g., decrease in the span between the upper hook and the lower hooks) owing to the resiliently flexible nature of the device 200. That is, once the upper hook 220 is in place, the upper hook 220 will contract, ensuring that the helmet 203 is securely fitted to the insect's head 910.

For example, a hook 220 at the upper part of the helmet 203 may flex away in response to the force and then flex back owing to the resiliently flexible nature of the helmet 203.

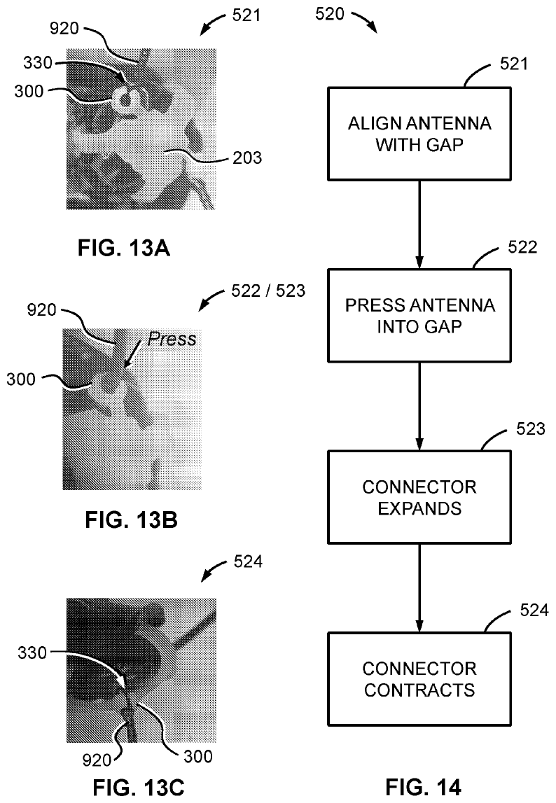

Once the helmet 203 is secured to the insect's head 910, the device 200 enables a relatively quick method of securing the connectors 300 to the antennae 920. A method 520 of attaching a connector 300 to an antenna 920 will be described with reference to the images of FIG. 13A to FIG. 13C and to the schematic diagram of FIG. 14.

In step 521 of the method 520, an antenna 920 is aligned with the gap 330 of the connector 300 (FIG. 13A). In some examples, this step is optional, e.g., the helmet 203 in a default state may be configured to position the gap 330 of the connector 300 in alignment with an antenna 920. Preferably, the connector 300 is positioned near the base of the antenna.

In step 522, the antenna 920 may be gently pressed toward and into the gap 330 (FIG. 13B). The pressing force on the antenna 920 causes the antenna 920 to force the gripping members of the connector 300 apart. The resiliently flexible nature of the material of the connector 300 will cause the gap to “expand”. That is, the gap 330 “expands” (step 523) or the gripping members flex to permit the antenna 920 to at least partially pass through the gap 330. The antenna 920 is preferably pushed until the antenna 920 passes through the gap 330 and makes contact with the inner surface of the connector 300.

The resiliently flexible nature of the connector 300 causes the gap 330 to “contract” (step 524) or to revert to its original size. The gripping members 314 tend to revert back to their default shape and/or position. The gripping members 314 receive the antenna and reverts elastically to at least partially “close” or narrow the gap 330.

As shown in FIG. 13C, the antenna 920 may be almost entirely bounded by the connector 300. The gap 330 is sized to be smaller than the diameter of the antenna. The antenna 920 will remain engaged within the connector 300 and will not easily move out through the gap 330. Owing to the resiliently flexible nature of the connector 300, the connector 300 can provide a tight grip on the antenna 920. The gripping members or the parts of the connector 300 surrounding the antenna 910 may exert pressure on the antenna's surface, ensuring a stable connection for neurostimulation or electrical contact. That is, the antenna 920 and the connector 300 (and hence the device 200) are secured together by a wholly mechanical coupling.

It can be understood from the foregoing description that various embodiments of the device 200, including the connectors 300, can be detachably coupled to an insect by a wholly mechanical coupling.

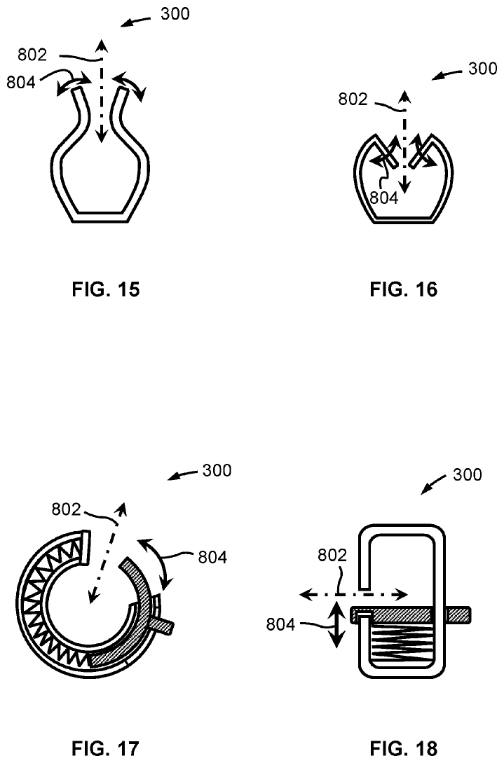

FIG. 15 and FIG. 16 are schematic illustrations of physical profiles or cross-sectional shapes that may confer the characteristics of resiliency in the connector 300. The connector distal ends 332 in each of the examples shown have a default connector state in which the connector distal ends 332 are biased to be closer to one another than in an expanded connector state in which the connector distal ends 332 have been forced further apart. In a connector default state, the resilient configuration of the connector 300 disposes the gripping members 314 to minimize the gap size or to close up the gap 330. In a connector expanded state, at least one of the gripping member 314 may be displaced to provide a larger gap size for the appendage 902 to pass through or pass partially through the gap 330. The gripping members 314 are biased toward one another and will grip the appendage 902 securely. In some examples, the gripping members cooperate to grip the appendage 902 from opposing sides of the appendage 902 such that the compressive forces or compressive action applied by the gripping members 314 hold the appendage 902 securely.

FIG. 17 and FIG. 18 are schematic illustrations showing other examples in which the displacement bringing the connector distal ends 332 closer to one another is enabled by a spring-loaded action. The connector 300 in these examples may include a spring 360 in abutment with at least one of the gripping member 314 (also referred to as the at least one spring-loaded gripping member). In a connector default state, the spring 360 biases the at least one spring-loaded gripping member 314 to minimize the gap size or to close up the slot gap. In a connector expanded state, the at least one spring-loaded member 314 may be displaced to provide a larger gap size for the appendage 902 to pass through or pass partially through the slot gap. The gripping members 314 are biased toward one another and will grip the appendage 902 securely.

As shown, the gripping members 314 may be any one of various angled configurations. In some embodiments, the gripping members 314 are symmetrically disposed relative to the connector securing member 312 (e.g., examples of FIG. 15 and FIG. 16). In some other embodiments, the gripping members 314 are asymmetrically disposed relative to the connector securing member 312 (e.g., example of FIG. 10B, FIG. 17, and FIG. 18). In some examples, one of the gripping members 314 has a wider range of movement compared to another one of the gripping members 314 (e.g., examples of FIG. 17, and FIG. 18).

Referring again to FIG. 10A and FIG. 10B, electrically conductive tapes or wires may be disposed on the connector 300. For example, an electrically conductive tape 144 may connect an electrode 150 to the rest of the controller system 100, in which the electrode 150 is disposed on a connector inner surface 340. In some embodiments, the electrode 150 at the connector 300 (also referred to as the connector electrode 152 for the sake of brevity) may extend along the connector inner surface 340 that corresponds to the connector securing member 312. In some embodiments, the connector electrode 152 may extend along the connector inner surface 340 that corresponds to at least one of the gripping members 314. In some preferred embodiments, the connector electrode 152 may extend along the connector inner surface 340 that corresponds to the connector securing member 312 and both the gripping members 314.

In the various examples provided above, when the appendage 902 is received or at least partially received between connector space 335 and/or the gap 330, the gripping members 314 will by default grip the appendage 902 securely. The connector electrode 152 may be correspondingly pressed into operable contact with neuromuscular sites of the appendage 902 by being in pressed abutment.

In some examples where the device 200 is coupled to the head 910 of the insect 900 and the connector 300 is coupled to an antenna 920, the connector 300 will be biased towards the head 910 (e.g., by the resiliently flexible biased members 224 that would be hooked about the head 910) simultaneously with the connector electrode 152 maintaining a tight grip on the antenna 920. The connector electrode 152 will be biased towards forming an operable contact with the antenna 920 at the root of the antenna 920.

As described above, an appendage 902 (such as an antenna 920) of the insect 900 may be coupled to the connector 300 by a clip-on action as the antenna 920 is passed through the gap 330 of the connector. The transverse displacement 802 involved in passing an appendage 902 through the gap 330 is much shorter than a longitudinal displacement 803 involved in threading the same appendage 902 through the connector space 335. Using the device 200, it is easier and faster to fasten a connector 300 to an antenna 920, and also less likely to injure the insect 900 in the course of the fastening process, as described above in respect of the device 200 in general.

In some embodiments, the connector 300 includes one gripping member 314 that is resiliently displaceable and biased toward narrowing or closing the gap 330. In some embodiments, the one or more gripping members 314 may be displaced by being pushed against or pressed against an appendage 902 of the insect 900, with the connector 300 being held in one hand by the user, and with the insect 900 being held in another hand by the same user. In these embodiments, the gripping members 314 are configured to be displaceable under a suitably light force so as to avoid injury to the insect 900. Advantageously, the connector 300 can be coupled to relatively fragile body parts of an insect 900 without surgery and without permanent injury.

### Kit

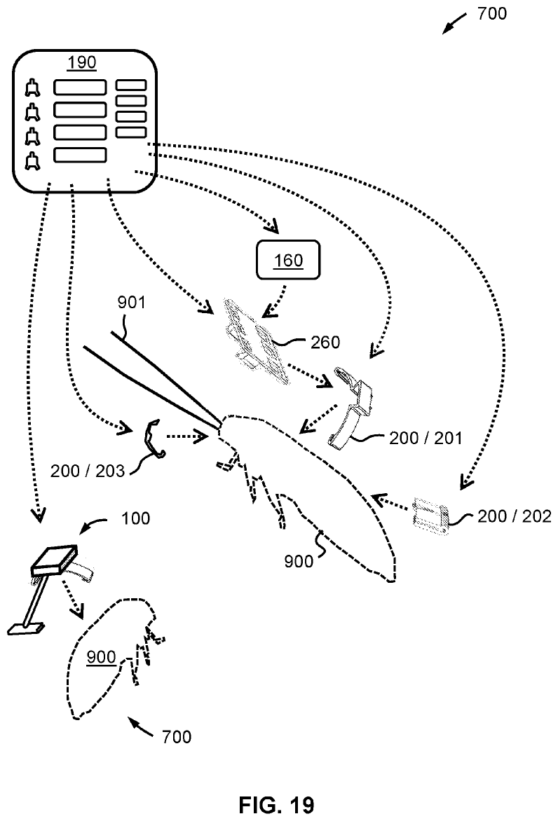

As surgical procedures would not be required if the device 200 is used, a user can equip an insect 900 with the controller system 100 (e.g., including a backpack 160 and a plurality of electrodes 150) in the field, e.g., outside the laboratory. According to one aspect of the present disclosure, as illustrated schematically in FIG. 19, a kit 190 can be provided for use in the field or on-site, in which the kit 190 includes a plurality of the device 200. The device 200 can advantageously be secured to the insect 900 more quickly and with less stress to the insect 900. The device 200 enables multiple units of the device to be coupled on/removed from one insect 900 quickly and easily (as compared to surgical procedures to implant the same number of electrodes).

One or more electrodes 150 may be disposed at the contact surface 240 (inner surface) of the base member 210. The resilient bias and/or the compressive action of the members is configured to put the electrodes 150 in pressed abutment with respective neuromuscular sites at the body part of the insect 900.

For some applications, the user can use the kit to quickly deploy a single insect-computer hybrid system 700, in which the insect-computer hybrid system 700 is an insect 900 equipped with the controller system 100 and the desired electrodes 150 suitably disposed at selected neuromuscular sites. The user can use the kit to attach the desired number and types of devices 100 to an insect, with the desired controller system 100, etc.

For example, an insect-computer hybrid system 700 may be deployed down in a small and long pipe to collect data from within the pipe.

For some applications, the user can use the kit to quickly deploy multiple insect-computer hybrid systems 700. Advantageously, the kit 190 can enable the user to deploy a team of insect-computer hybrid systems 700 of various sizes, various types of insects 900, and/or with controller systems 100 pre-programmed for various functions. The user can equip a plurality of insects 900 with electrodes 150 and/or backpacks 160 in the field immediately before releasing the insects 900 to carry out their mission). When the equipped insects 900 are retrieved (e.g., after completing their mission), the devices 200 can be removed from the insects 900. The insects 900 can benefit from the shorter period of time in the equipped condition.

The user can equip more insects or fewer insects 900 with differently programmed backpacks 160 or controller systems 100 in response to the actual needs of the situation in the field.

Beneficially, the insects are not irreversibly harmed by the process to attach/detach the electrodes 150 to/from the insect 900. The backpacks 160 and the electrodes 150 can be attached/detached to/from the insect along with the devices 200 in the same action. The mechanical mechanism for attachment of the device 200 also provides a clean, mechanical detachment that does not require the use of solvents to dissolve adhesives. Use of the device 200 would be kinder to the insects as some solvents may also remove the protective wax-like coating on the exoskeleton.

The kit may include a plurality of the device 200 in a range of sizes, shapes and/or configurations, to suit insects of different sizes and/or shapes. The plurality of device 200 may be provided as separate articles to enable mix-and-match of the devices. For convenience and efficiency, the kit 190 may include pre-assembled modules of the controller system 100, e.g., with a backpack 160 and wiring interconnecting two or more devices 200 for snapping onto one insect 900. In some examples, the kit 190 may include a range of devices 200 suitable for detachable coupling to the thorax 930, abdomen 940, or cerci 950 of the insect 900. In some examples, the kit may include a range of devices 200 suitable for detachable coupling to the head 910 of the insect 900 or, more specifically, to the antennae 920 of the insect 900. For example, the kit 190 may include various embodiments of the device 200, including devices 200 for detachable mechanical clip-on coupling with body parts with a relatively larger cross-sectional area, such as but not limited to the thorax or the abdomen of the insect. For example, the kit 190 may include various embodiments of the device 200, including device 200 for detachable mechanical clip-on coupling with body parts with a relatively smaller cross-sectional area, such as but not limited to appendages such as the antennae.

According to various embodiments of the present disclosure, a device for use with an insect includes: a base member; and two or more hooks, the two or more hooks being disposed at different sides of the base member, the two or more hooks each being a resiliently flexible hook.

The device may be detachably attachable to the insect by a wholly mechanical coupling of the two or more hooks with the insect.

The two or more hooks may each include: a securing member, the securing member being coupled with the base member; a biased member; and an angled element, the angled element coupling the securing member and the biased member to define a slot between the securing member and the biased member.

Each of the two or more hooks may be resiliently flexible to provide a variable slot height between the securing member and the biased member.

The device may further include a serrated surface, the serrated surface being disposed on at least one of the securing member and the biased member.

The device may further include an electrode, the electrode being disposed at an inner surface of the device.

The electrode may include a surface electrode. The device in a wholly mechanical coupling with the insect provides the surface electrode in pressed abutment with a neuromuscular site of the insect.

The securing member and the biased member may define respective axes that are parallel and non-coincidental to one another in a default state.

The two or more hooks may be oriented in a similar direction.

The device is resiliently flexible to provide a variable span between two opposable hooks, the two opposable hooks being selected ones of the two or more hooks.

The two opposable hooks may be oriented in opposite directions to one another.

The device may include a backpack coupled to the base member, the backpack including a controller.

The device may include a mounting platform that is detachably coupleable with the base member.

The backpack may further include any one or more of the following: a communications module; a battery pack, and a power source.

The securing member and the biased member may define respective axes that are non-parallel to one another.

The device may be suitable for use with an appendage of the insect. The device may further include a connector. The connector may include: a connector securing member; and two gripping members. The two gripping members may extend from the connector securing member in different directions and ending in respective connector distal ends, the connector distal ends may be spaced apart from one another, in which the two gripping members are angled to dispose the connector distal ends in opposing orientations to define a gap therebetween, and in which the two gripping members are resiliently biased toward narrowing the gap.

The device may further include at least one connector electrode disposed on the at least one connector, in which in assembly, the appendage is in pressed abutment with the at least one connector electrode.

The gripping member may be spring-loaded to resiliently bias the gripping member to decrease a gap width of the gap.

The connector may be detachably attachable to the appendage by a wholly mechanical coupling.

A kit for use with one or more insects may include: a plurality of the device according to any described above, the plurality of the device being of various sizes for use with the one or more insects of various sizes.

An insect-computer hybrid system may include: an insect; and one or more devices, the one or more devices each according to any as described above, the one or more devices being detachably attached to a respective body part of the insect by a wholly mechanical coupling.

The at least one of the one or more devices may further include one or more electrodes, each of the one or more electrodes being in operable contact with a respective neuromuscular site of the insect.

A selected one of the one or more devices may be coupled to one of the head, the thorax, the abdomen, and the cercus of the insect.

All examples described herein, whether of apparatus, methods, materials, or products, are presented for the purpose of illustration and to aid understanding, and are not intended to be limiting or exhaustive. Modifications not involving inventive effort may be made by one of ordinary skill in the art without departing from the scope of the claimed invention.

1. A device attachable to and for use with an insect, comprising:
a base member; and
two or more hooks, the two or more hooks being disposed at different sides of the base member, the two or more hooks each being a resiliently flexible hook.

2. The device as recited in claim 1, wherein the device is detachably attachable to the insect by a wholly mechanical coupling of the two or more hooks with the insect.

3. The device as recited in claim 1, wherein the two or more hooks each comprises:
a securing member, the securing member being coupled with the base member;
a biased member; and
an angled element, the angled element coupling the securing member and the biased member to define a slot between the securing member and the biased member.

4. The device as recited in claim 3, wherein each of the two or more hooks is resiliently flexible to provide a variable slot height between the securing member and the biased member.

5. The device as recited in claim 3, further comprising a serrated surface, the serrated surface being disposed on at least one of the securing member and the biased member.

6. The device as recited in claim 1, further comprising an electrode, the electrode being disposed at an inner surface of the device.

7. The device as recited in claim 6, wherein the electrode comprises a surface electrode, wherein the device in a wholly mechanical coupling with the insect provides the surface electrode in a pressed abutment with a neuromuscular site of the insect.

8. The device as recited in claim 3, wherein the securing member and the biased member define respective axes that are parallel and non-coincidental to one another in a default state.

9. The device as recited in claim 8, wherein the two or more hooks are oriented in a similar direction.

10. The device as recited in claim 1, wherein the device is resiliently flexible to provide a variable span between two opposable hooks, the two opposable hooks being selected ones of the two or more hooks.

11. The device as recited in claim 10, wherein the two opposable hooks are oriented in opposite directions to one another, wherein the securing member and the biased member define respective axes that are non-parallel to one another.

12. The device as recited in claim 1, further comprising a backpack coupled to the base member, the backpack including a controller and any one or more of the following: a communications module; a battery pack, and a power source.

13. The device as recited in claim 12, further comprising a mounting platform, the mounting platform being detachably coupleable with the base member.

14. (canceled)

15. (canceled)

16. The device as recited in claim 1 for use with an appendage of the insect, further comprising:
a connector, the connector including:
a connector securing member; and
two gripping members, the two gripping members extending from the connector securing member in different directions and ending in respective connector distal ends, the connector distal ends being spaced apart from one another,
wherein the two gripping members are angled to dispose the connector distal ends in opposing orientations to define a gap therebetween,
and wherein the two gripping members are resiliently biased toward narrowing the gap.

17. The device as recited in claim 16, further comprising at least one connector electrode disposed on the at least one connector, wherein in assembly, the appendage is in pressed abutment with the at least one connector electrode.

18. The device as recited in claim 16, wherein the gripping member is spring-loaded to resiliently bias the gripping member to decrease a gap width of the gap.

19. The device as recited in claim 16, wherein the connector is detachably attachable to the appendage by a wholly mechanical coupling.

20. A kit for use with one or more insects, comprising:
a plurality of the device according to claim 1, the plurality of the device being of various sizes for use with the one or more insects of various sizes.

21. An insect-computer hybrid system, comprising:
an insect; and
one or more devices, the one or more devices each as recited in claim 1 and detachably attached to a respective body part of the insect by a wholly mechanical coupling.

22. The insect-computer hybrid system as recited in claim 21, wherein at least one of the one or more devices further comprises one or more electrodes, each of the one or more electrodes being in operable contact with a respective neuromuscular site of the insect, the respective body part of the insect being selected from the following: a head, a thorax, an abdomen, and a cercus of the insect.

23. (canceled)
